(19) United States
(12) Patent Application Publication
(10) Pub. No.: US 2013/0096123 A1
(43) Pub. Date: Apr. 18, 2013

(54) RADIATION SENSITISER COMPOSITIONS

(71) Applicant: Malvin Leonard Eutick, Northbridge (AU)

(72) Inventor: Malvin Leonard Eutick, Northbridge (AU)

(73) Assignee: EUPHARMA PTY LTD, Northbridge (AU)

(21) Appl. No.: 13/640,975

(22) PCT Filed: Apr. 14, 2011
(86) PCT No.: PCT/AU2011/000433

§ 371 Date: Dec. 26, 2012

(30) Foreign Application Priority Data
Apr. 14, 2010 (AU) 2010901579
Nov. 9, 2010 (AU) 2010904970

(51) Int. Cl. A61K 9/00

(52) U.S. Cl. CPC A61K 9/0053
USPC 514/235.8

(57) ABSTRACT

The invention provides an oral liquid composition of a hypoxic radiation sensitiser comprising the radiation sensitiser in a concentration of greater than 5 mg/ml, and a method of treating a subject with a hypoxic condition comprising administration to the subject of a combination of a composition of the invention and radiation.

### FIELD OF INVENTION

This invention relates to compositions of radiation sensitisers and methods of administration of those compositions. In particular the invention relates to an oral liquid composition of a hypoxic radiation sensitiser, and a method of oral administration of a liquid composition of a hypoxic radiation sensitiser.

### BACKGROUND

In many malignancies, the cancerous cells experience a significant reduction in oxygen availability due to poor vascularisation resulting from their rapid growth, leading to intratumoral hypoxia. Intratumoral hypoxia reduces the efficacy of radiation therapy, due to reduced radiosensitivity of the hypoxic cells. It is suggested (Rowinsky E K. Oncology, 1999: 13(105), that under anaerobic conditions the radiation dose may have to be increased by a factor of 2.5 to 3 times to achieve the same degree of cytotoxicity that occurs under oxygenated conditions.

Hypoxia is a common feature of solid tumours and generally occurs when they develop over 100 to 150 μm away from functional blood vessels (Helmlinger G, et al. Nat Med. 1997 3:177-182). This hypoxia is widespread in not only primary malignancies but also in their metastases. This usually results in an intratumoral oxygen tension of 0-20 mmHg compared with levels from 24-66 mm Hg in normal human tissues (Brizel D M, et al Int J Radiat Oncol Biol Phys 1995, 32: 1121125).

Retrospective studies in malignancies have determined that poor tumour oxygenation is the strongest prognostic indicator of radiotherapy treatment outcomes (Gatenby R A, et al. Int. J Radiat Biol Phys, 1988, 14: 831-838; Hockel M et al Cancer Res, 1996 56:4509-4515; Brizel D M, et al Int J Radiat Oncol Biol Phys 1997, 38:285-289).

In these circumstances then a potential exists to enhance the efficacy of a radiation dose and/or to moderate the development of radiation toxicity by increasing the oxygenation levels of tumour cells within the solid tumour mass prior to radiation treatment Radiation sensitisers enhance the tissue response to radiation due generally by mimicking the effects of oxygen, which induces the formation and stabilisation of toxic DNA radicals (Rowinsky E K. Oncology, 1999: 13(105).

The use of radiation sensitisers has been examined to this end for solid tumours but in many instances their toxicity (especially neurotoxicity) or lack of efficacy or poor patient tolerance due to the large doses required has reduced their treatment efficacy. The issue of the dose size is a primary concern for treatment as usually the amount of drug to be taken on a daily basis is approximately 1000 mg (or greater). This large dose has been given via tablets and capsules. This presents a significant obstacle to patient compliance as many suffer mucositis or damage to the surfaces of their mucosa due to the radiation effects thus limiting their ability to swallow the large solid doses.

Such amenable malignancies include head and neck cancers (including carcinoma of the larynx, glottis and oesophagus), adrenocarcinoma of the pancreas, gastrointestinal cancers, breast cancers, uterine and cervical cancers, lung cancers, malignant glioma, colorectal cancers, prostate cancer, kidney and bladder cancer, squamous cell carcinomas, melanoma, glioblastoma, and solid tumours where the hypoxia is related to blood vessels being greater than about 100 to 150 μm from a cell within the tumour.

A radiation sensitiser can be defined as an agent that increases cell susceptibility to ionising radiation. A hypoxic radiation sensitiser is an agent which increases cell susceptibility to ionising radiation where the cancer being treated is a hypoxic cancer, that is a cancer causing intratumoral hypoxia as described above.

Radiation sensitisers act in a number of ways to make cancer cells more susceptible to death by radiation than surrounding normal cells, and several such compounds have been investigated for the treatment of solid tumors (Lawrence T S, Oncology (Williston Park). 2003 December; 17 (12 Suppl 13):23-8). Examples of such agents are nitroimidazoles such as misonidazole, metronidazole, tinidazole, sanazole nimorazole and etanidazole as well as other unrelated compounds such as tirapazamine, gadolinium texaphyrin.

A group of compounds of particular interest as radiation sensitisers is nitroaromatic and nitroheterocyclic compounds. These compounds were originally used primarily as anthelmintics, but have found application or been examined as radiation sensitisers in the treatment of hypoxic cancers such as pancreatic and head and neck cancers which are generally fatal.

This application of a radiation sensitiser combined with radiation therapy also improves outcomes for patients who may have no or low tolerance for some of the more conventional chemotherapeutic agents, such as cisplatin.

Presently radiation sensitisers are occasionally administered intravenously or more commonly orally as tablets or capsules, depending on their pharmacokinetics and solubility and the amount of drug that has to be administered. Traditional radiation sensitisers such as misonidazole and nimorazole require large doses of drug for efficacy—in the order of 0.5 g to 2.5 g per day prior to radiation therapy. The therapy usually lasts 5 to 6 days and is then followed by a further series of multiple radiation cycles.

Large physical tablets or capsules have to be used to administer such large doses of the sensitiser, which are difficult, and sometimes impossible, for the patient to swallow (J. Overgaard et al., J Radiotherapy and Oncology 46 (1998) 135-146). Radiation of many hypoxic cancers, for example and particularly upper body cancers, result in damage to the salivary glands and mucosa which adversely and severely (especially in the later cycles of treatment) affects swallowing ability, further exacerbating the problem of administration of these agents as tablets or capsules.

In addition, many radiation sensitisers have limited solubility and are therefore not available for intravenous administration.

Therefore, there would be an advantage in a formulation of hypoxic radiation sensitisers which may overcome at least some of the above-mentioned disadvantages or provide a useful or commercial choice.

### SUMMARY OF THE INVENTION

In one form, although it need not be the only or indeed the broadest form, the invention resides in an oral liquid composition of a hypoxic radiation sensitiser comprising the radiation sensitiser in a concentration of greater than 5 mg/ml.

In another form, the invention resides in a method of treating a patient suffering from a hypoxic cancer comprising oral administration of a liquid composition of a hypoxic radiation sensitiser and radiotherapy to the patient, wherein the liquid composition comprises the radiation sensitiser in a concentration of greater than 5 mg/ml.

In a further form, the invention resides in use of an oral liquid of a hypoxic radiation sensitiser of concentration greater than 5 mg/ml in conjunction with radiotherapy to treat hypoxic cancer.

In a further form, the invention provides a kit comprising a powder formulation or solid blend of at least one hypoxic radiation sensitiser, or a concentrated solution of at least one radiation sensitiser, and a diluent and/or vehicle, wherein the kit is used to assemble an oral liquid composition of a hypoxic radiation sensitiser. This kit may also contain a holding or administration device for oral administration of the sensitiser.

Preferably the liquid composition is a solution of at least one radiation sensitiser, or a suspension of at least one radiation sensitiser in a pharmaceutically acceptable carrier, to a concentration of greater than 5 mg/ml.

Preferred radiation sensitisers are nitroaromatic and nitroheterocyclic compounds, especially the substituted 2-nitroimadazoles, 4-nitroimadazoles and 5-nitroimadazoles including: azomycin, Imuran, misonidazole, metronidazole, isometronidazole, tinidazole, pimonidazole, nimorazole, secnidazole, dimetridazole, ternidazole, 1-methyl-2-(p-fluorophenyl)-5-nitroimadazole, flunidazole, chlomizole, ronidazole, panidazole, ornidazole, nitroimadazole thiadiazole, benznidazole, 5-isopropyl-1-methyl-2-nitroimadazole, 2-methyl-5-nitroimadazole-1-ethanol methanesulfonate, bamnidazole, 3a,4,5,6,7,7a-hexahydro-3-(1-methyl-5-nitroimadazol-2-yl)-1,2-benzisoxazole, carnidazole, sulnidazole, moxnidazole, etanidazole, doranidazole, azanidazole, omidazole, propenidazole, nitrefazole, etanidazole, sanazole, 2-amino-4-(2-ethynyl-1-methyl-5-nitroimadazole)-pyrimidine, 1,4-bis(1-methyl-5-nitroimidazolyl-(2-methylenimino))piperazine, pirinidazole, microprofen, satranidazole, 3α,4,5,6,7,8,9,9α-octahydro-3-(1-methyl-5-nitroimidazol-2-yl)cycloocta(d) isoxazol, fexinidazole, tivanidazole, abunidazole, and 1-(2-fluoroethyl)-2-nitroimidazole, 1-(2-phenoxyethyl)-2-nitroimidazole, 1-(4-iodophenoxypropyl)-2-nitroimidazole.

Also preferred are derivatives of the foregoing, including ring substituted derivatives, such as lower alkyl substitutions, (suitable examples include methyl, ethyl, propyl, butyl, and isomers thereof), aryl substitutions (phenyls or heterocylic ring structures unsubstituted or substituted by lower alkyl, halogens, hydroxyl, lower alkoxy, nitro, amino, monoalkyl or dialkylamino and the like) and also including thiols, diols, diones, metal complexes, aziridino derivatives, halogenated derivatives such as fluorinated, iodinated and brominated structures.

Also preferred are pharmaceutically acceptable salts of the foregoing, such as hydrochlorides, fumarates, phosphates, sulphates, nitrates, sulphonates, adipates, benzoates, citrates, gentisates, glutarates, glycolates, hippurates, lactates, maleates, malates, xinafoates, nicotinates, succinates, tartrates, aspartates, glutamates, mesylates, tosylates, ascorbates, acetonides, and saccharinates.

Also included are hydrates, crystal polymorphs of each or any of the foregoing, single isomers, enantiomers and mixtures thereof.

Preferably the concentration of the hypoxic radiation sensitiser is greater than 5 mg/ml. The concentration can also be greater than 6 mg/ml, greater than 10 mg/ml, greater than 20 mg/ml, greater than 50 mg/ml, greater than 100 mg/ml, greater than 200 mg/ml, greater than 500 mg/ml, greater than 1000 mg/ml, or greater than 1500 mg/ml.

### DETAILED DESCRIPTION OF THE INVENTION

Referring to the aspects of the invention summarised above, the oral liquid composition of the invention can be made in any practicable manner. The invention can also take any practicable form appropriate for oral administration of a liquid composition of the invention. For example, the composition can be a solution of at least one radiation sensitiser, or a suspension of at least one radiation sensitiser in a suitable carrier.

The vehicle or diluent for the oral liquid composition of the invention can be any pharmaceutically acceptable vehicle, for example water, lipids, lipoidal structures, pharmaceutically acceptable oils, or mixtures thereof, including for example parabens, glycols, cellulose derivatives.

The concentration of the hypoxic radiation sensitiser in the oral liquid composition of the invention is generally from 5 mg/ml to 1500 mg/ml.

The hypoxic radiation sensitiser may be used in any crystalline or powdered form that allows it to be prepared as a liquid composition for oral administration. The hypoxic radiation sensitiser may also be micronized to a size suitable for more effective solubilisation or suspension, and/or other processing to improve the properties of the particles. Generally micronisation reduces the particle size to between 1 μm and 15 μm, and more specifically about 80% of the particles between 1 μm and 15 μm, or about 50% of the particles between 1 μm and 10 μm, or about 25% of the particles between 1 μm and 5 μm. Effective micronisation of the compound crystals reduces abrasion of the particles on administration and swallowing, and eases throat passage of a suspension of the invention.

In preparation of an oral liquid solution of the invention, the properties of the particles of hypoxic radiation sensitiser can be improved for solubility, taste and/or bioavailability. This can be achieved by, for example complexing, encasing, encapsulating or binding the particles into a carrier. These improvements can be achieved using compounds and processes known in the art, including for example, alpha, beta or gamma cyclodextrins, and their many derivatives such as methylated beta-cyclodextrins, hydroxypropyl beta cyclodextrin and sulfobutyl ester beta-cyclodextrin and mixtures of any of this class of compounds. Cyclodextrins or their derivatives are used generally from 1:0.01 to 1:100 molar ratio of the radiation sensitiser to cyclodextrin, preferably between 1:0.1 and 1:25. In addition to the cyclodextrins, the particles can also be encapsulated or complexed with, for example, chitin, lipophilic agents such as stearates, oleates and their esters and phospholipids such as egg phosphatidylethanolamine and polyvinyl acetate to more complex “block” type polymers such as polyethylene glycol and polylactic acid and poloxamer combinations of polyethylene oxide and polypropylene oxide type polymers,

Improvements in solubility and/or suspension properties of the particles radiation sensitiser can also be achieved using hyaluronic acid, lipid micelles, liposomes and/or cellulose.

A solution of the invention can also be in the form of a ‘pre-solution’ for further dilution and/or modification, for example by complexing, encasing, encapsulating or binding.

Passage of an oral liquid solution of the invention down the throat of the patient can be eased by addition of a surfactant or greasing agent or gel to the solution, such as detergents such as polysorbates, carboxymethylcellulose and its derivatives such as hydroxypropyl carboxymethylcellulose, polyethylene glycols, polyvinyl-pyrrolidone (PVP), sodium lauryl sulphate or phospholipids.

Passage of a suspension of the invention can also be eased by coating the hypoxic radiation sensitiser particles. The coating can be any suitable substance as described earlier, for example, lipid micelles, liposomes and/or cellulose. The particles can also be encapsulated for example with chitin, lipophilic agents such as stearates, oleates and their esters, methacrylates or PVP or PVA. Passage can also be eased by addition of a greasing agent or gel to the suspension, such as carboxymethylcellulose or PVP (polyvinyl pyrrolidone) or a phospholipid. The inclusion of the additives described above also facilitates administration of the suspension directly to the gut using nasogastric tubes if the throat of the patient does not allow swallowing.

Passage of oral liquid compositions of the invention may also be aided by using benzyl alcohol as a preservative since this molecule also has mild detergent and anaesthetic properties.

Mixtures of the above modifying compounds and processes can also be used for improving solubility of the hypoxic radiation sensitiser for solutions of the invention, for example by using cyclodextrins as the primary solubiliser and taste masking agent, but increasing its effectiveness with addition of other polymers and/or hydroxyacids and/or rheological agents, for example water soluble cellulose derivatives, hydroxypropyl methyl cellulose, polyvinylpyrrolidine, citric acid, malic acid and tartaric acid.

Ammonium salts such as ammonium hydroxide, and other ionic modifiers may also be used to enhance the complexation efficiency.

pH modification can also be effected to ensure the desired ionicity of the preparation and for patient comfort and compliance, since many patients during radiation cycles develop mucositis leaving their mucosa severely damaged thus exposing them to pain and burning sensations when taking oral liquids of high or low pH.

The inclusion of the additives described above also facilitates administration of the oral composition of the invention directly to the gut using nasogastric tubes if the throat of the patient is resistant to swallowing or too painful to swallow due to mucositis. Some of the additional components may also aid rapid absorption from the stomach or intestinal tract where an early peak drug concentration (C-max) is required prior to radiological treatment. For example, complexation with the cyclodextrins or their derivatives allows choice of fast desorption for a rapid Cmax, or a long period of drug absorption, effectively providing a slow release system.

An oral liquid composition of the invention can also include pharmaceutically appropriate stabilising and solubilising agents or agents that may also improve taste and bioavailability, such as agar, alginate, carboxymethylcellulose and its derivatives (hydroxypropylmethylcellulose), dextrates, pectin, polyethylene glycol, substituted polyethylene glycols such as the dicaprylocaprate esters, triglycerides, glycerol esters (monolinoleates and monooleates). Lubricants and surfactants can also be included, for example polyvinyl alcohol, castor oil or esters thereof, polysorbates, polydextrose and poloxamers. Gelling agents can also be included, such as the Carbomer polymers. In addition, dispersants such as gelatin and lecithin can be included, and stabilisers and antioxidants such as sodium bisulphate, ascorbic acid, and edetates. Osmotic agents such as mannitol and sodium chloride can also be included in the liquid composition of the invention.

Additionally, any polymer, sugar, polyhydric alcohol, salt, salt combination, aqueous solvent and mixed aqueous solvent and non aqueous solvent and the like, may be employed as a solubilising adjunct for the liquid composition of the invention, if the hypoxic radiation sensitiser is biocompatible with the desired product stability, as is known to a person skilled in the art.

The oral liquid composition of the invention can also include comfort enhancing agents such acids or alkalis to adjust the pH of the final composition to that of the bucal cavity, being pH 6-7, or buffers to allow the composition to be adjusted and held at the pH of the mouth or bucal cavity. These agents may also be used to maintain the stability of the sensitiser in liquid composition. Non limiting examples of pH modifiers, buffers and stabilisers include citric acid, tartaric acid, succinic acid, glutamic acid, ascorbic acid, lactic acid, acetic acid, malic acid, maleic acid, phosphates and sodium salts thereof, sodium or potassium hydroxide, sodium carbonate, sodium bicarbonate, mineral acids such as hydrochloric and sulphuric acids, tris buffer, meglumine, amino acids and their salts, and mixtures thereof. Such pH modifiers and stabilisers maintain a desired pH between 2 and 10, or between 2.5 and 10 in the solution.

An oral liquid composition of the invention can also include pharmaceutically appropriate flavours and sweeteners to mask or improve the taste and organoleptic properties of the hypoxic radiation sensitiser, if necessary. This facilitates tasting and swallowing of an oral solution, and improves patient compliance. Examples of such flavourants are vanilla, orange and lemon, mint, peppermint, chocolate, coffee flavour, cherry, strawberry and the like, and sweeteners such as sugar, sucralose, fructose, saccharin, aspartame, cyclamate, acesulfame potassium, xylitol, sorbitol, and delayed sweeteners such as mono-ammonium glcyrrhizinate and other sugars and sweetening agents, and taste enhancers and modifiers and masking agents such as citric acid, and clove oil.

An oral liquid composition of the invention can be formulated as a single dose, multidose, or can be provided in a kit comprising a container, for example a sachet, of the complexed hypoxic radiation sensitiser, and a mixing container containing the vehicle, optionally including additives as discussed above. The single dose can be provided in any practicable form, including as a pre-mixed sachet, or on-site mixable kit, including optional additives as discussed above.

When an oral liquid composition of the invention is formulated as a multidose formulation, a pharmaceutically appropriate preservative or mixture of preservatives can be added to the solution, such as benzoates, sorbates, benzyl alcohol, hydroxybenzoates (parabens), phenoxyethanol, quaternary ammonium salts such as benzalkonium chloride, sodium bisulphate, and ethanol.

The oral liquid compositions of the invention can optionally include, in addition to the hypoxic radiation sensitiser, the following classes of drugs for the purposes as indicated. Inclusion of any one of, or a combination of any of these drugs enhances the usefulness of the oral composition of the invention and/or improves the administration experience of the patient:
Anti-inflammatory drugs such as benzydamine, ibuprofen, paracetamol, and other non steroidal anti-inflammatory drugs (NSAIDs) and derivatives thereof;
drugs that stop the throat being infected such as chlorhexidine, cetyl alcohol; and ethanol;
topical anaesthetics such as lignocaine (lidocaine), oxetacaine, bupivacaine, ropivocaine, mepivacaine, and dyclonine;
drugs that may calm the patient such as anti-anxiolytics and sedatives, or that induce some conscious sedation of the patient such as midazolam and other benzodiazepines and etifoxine;
drugs that may reduce the nausea such as metoclopramide, ondansetron, granisetron, droperidol, and dexmedetomidine;
non-NSAID pain relieving drugs such as fentanyl and its derivatives, opioids such as morphine, oxycodone, hydromorphone, nalbuphine and codeine and other pain relieving substances such as nefopam;
drugs that may act to reduce the a state of depression, including the SSRI's and MAO inhibitors such as amytriptyline;
topically acting corticosteroid drugs such as hydrocortisone, triamcinolone acetonide or flucinolone; and/or
Drugs that may reduce the toxicity by shortening the half life of the radiation sensitiser such as phenytoin and phenobarbitone.

The hypoxic radiation sensitiser in an oral liquid composition of the invention can be any hypoxic radiation sensitiser. This may be utilised in its normal crystalline form but can also be in a form which is be suitable for micronisation and dissolution. Preferably the hypoxic radiation sensitiser is a nitroimidazole salt, as exemplified in the list provided above.

Detailed, non-limiting examples of the invention are provided.

### A. Examples of Suspensions of the Invention

### Example 1

### Water Based Suspension with Taste Masking

An oral suspension of a hypoxic radiation sensitiser of the invention is prepared as follows:

| | |
|---|---|
| Nimorazole (250 mg/ml) | 25 g |
| Fructose (500 mg/ml) | 50 g |
| Sucralose (5 mg/ml) | 0.5 g |
| Orange oil (2 mg/ml) | 0.2 g |
| Clove Oil (1 mg/ml) | 0.1 g |
| Sodium benzoate (1 mg/ml) | 0.1 g |
| Water to 100 ml | |

The sodium benzoate is dissolved in warm (60-80° C.) purified water for 30 minutes;
fructose and sucralose are added slowly with constant stirring until dissolved (about 60 minutes);
orange oil and clove oil are added and the mixture is stirred for a further 30 minutes;
the mixture is cooled to 20-25° C.;
nimorazole is added and the suspension is stirred for a further 60 minutes or until homogenous;
pH is adjusted to about 3.5-4.0 with citric acid and the volume is made up to 100 ml with stirring.

### Example 2

### Water Based Suspension with Taste Masking, Lubricant and Viscosing Agent

A second oral suspension of a hypoxic radiation sensitiser of the invention is prepared as follows:

| | |
|---|---|
| Nimorazole (250 mg/ml) | 25 g |
| Hydroxypropyl methyl cellulose (HPMC) (5 mg/ml) | 0.5 g |
| Fructose (500 mg/ml) | 50 g |
| Sucralose (5 mg/ml) | 0.5 g |
| Orange oil (2 mg/ml) | 0.2 g |
| Clove Oil (1 mg/ml) | 0.1 g |
| Sodium benzoate (1 mg/ml) | 0.1 g |
| Water to 100 ml | |

HPMC is dissolved by slowly adding to water at 80° C. with stirring for 60 minutes;
sodium benzoate is dissolved by adding to and stirring in the warm mixture (60-80° C.) for 30 minutes;
fructose and sucralose are added slowly with constant stirring until dissolved (about 60 minutes);
orange oil and clove oil are added and the mixture is stirred for a further 30 minutes;
the mixture is cooled to 20-25° C.;
nimorazole is added and the suspension is stirred for a further 60 minutes or until homogenous;
pH is adjusted to about 3.5-4.0 with citric acid and the volume is made up to 100 ml with stirring.

### Example 3

### Water Based Suspension with Taste Masking and Emulsifier/Surfactant

A third oral suspension of a hypoxic radiation sensitiser of the invention is prepared as follows:

| | |
|---|---|
| Nimorazole (250 mg/ml) | 25 g |
| Polysorbate 20 (Tween 20) (0.5 mg/ml) | 0.5 g |
| Fructose (500 mg/ml) | 50 g |
| Sucralose (5 mg/ml) | 0.5 g |
| Orange oil (2 mg/ml) | 0.2 g |
| Clove Oil (1 mg/ml) | 0.1 g |
| Sodium benzoate (1 mg/ml) | 0.1 g |
| Water to 100 ml | |

Polysorbate 20 is dissolved by slowly adding to water at 80° C. with stirring for 60 minutes;
sodium benzoate is dissolved by adding to and stirring in the warm mixture (60-80° C.) for 30 minutes;
fructose and sucralose are added slowly with constant stirring until dissolved (about 60 minutes);
orange oil and clove oil are added and the mixture is stirred for a further 30 minutes;
the mixture is cooled to 20-25° C.;
nimorazole is added and the suspension is stirred for a further 60 minutes or until homogenous.
pH is adjusted to about 3.5-4.0 with citric acid and the volume is made up to 100 ml with stirring.

The Polysorbate 20 can be substituted with Polysorbate 80 and the level of suspension altered by varying the concentration of the Polysorbates.

### Example 4

### Water Based Suspension with Taste Masking and Emulsifier/Surfactant

A fourth oral suspension of a hypoxic radiation sensitiser of the invention is prepared as follows:

| | |
|---|---|
| Nimorazole (250 mg/ml) | 25 g |
| Polysorbate 20 (Tween 20) (0.5 mg/ml) | 0.5 g |
| Poloxamer 407 (1.5 mg/ml) | 1.5 g |
| Fructose (500 mg/ml) | 50 g |
| Sucralose (5 mg/ml) | 0.5 g |
| Orange oil (2 mg/ml) | 0.2 g |
| Clove Oil (1 mg/ml) | 0.1 g |
| Sodium benzoate (1 mg/ml) | 0.1 g |
| Water to 100 ml | |

Polysorbate 20 is dissolved by slowly adding to water at 80° C. with stirring for 60 minutes;
sodium benzoate is dissolved by adding to and stirring in the warm mixture (60-80° C.) for 30 minutes;
Polyoxomer 407 is dissolved in the mixture by slow addition with stirring to the mixture;
fructose and sucralose are added slowly with constant stirring until dissolved (about 60 minutes);
orange oil and clove oil are added and the mixture is stirred for a further 30 minutes;
the mixture is cooled to 20-25° C.;
nimorazole is added and the suspension is stirred for a further 60 minutes or until homogenous.
pH is adjusted to about 3.5-4.0 with citric acid and the volume is made up to 100 ml with stirring.

The Polysorbate 20 can be substituted with Polysorbate 80 and the level of suspension altered by varying the concentration of the polysorbates.

Poloxamers are surfactants of the poly(oxyethylene)poly(oxypropylene) copolymer type, commonly used in the pharmaceutical field. A preferred poloxamer is poloxamer 407—a poly(oxyethylene)poly(oxy-propylene) copolymer wherein the polyoxypropylene portion has an average molecular weight of about 4000 and the polyoxyethylene portion amounts to 70% by weight. Other suspending agents such as polyvinyl pyrrolidine (PVP) may be used in place of the HPMC or polysorbates.

In these water based formulations the preservative sodium benzoate may be substituted with hydroxy benzoates (parabens) appropriate to obtain a preservative effect and to lift the pH to a more acceptable one with the bucal cavity (between 6 and 7). A pH adjustment may utilise sodium or potassium hydroxide.

The flavour additive of the suspension may be changed as desired to mint, vanilla, chocolate, lemon or other natural or synthetic flavourants. Citric acid can be added to obtain a fresh flavour effect and reduce bitterness.

A sweet taste can be obtained by addition of artificial sweeteners such as aspartame, saccharin cyclamate, acesulfame potassium or natural sugars such as sucrose, glucose, fructose, sorbitol, xylitol, maltodextrin and delayed sweeteners such as mono-ammonium glcyrrhizinate.

Other active pharmaceuticals can be added to the above formulations, for example:
Topical anaesthetics eg benzydamine (0.15% w/v) or
lignocaine 0.33% to 2% w/v or
dyclonine, benzyl alcohol 0.9% w/v;
Anti-inflammatory agents eg flurbiprofen or ketoprofen or ibuprofen;
Disinfectants eg cetylpyridinium chloride 0.1% w/v.

### Example 5

### Propylene Glycol (PG) Based Suspension with Taste Masking

A fifth oral suspension of a hypoxic radiation sensitiser of the invention is prepared as follows:

| | | |
|---|---|---|
| Nimorazole (250 mg/ml) | 25 | g |
| Ethanol | 2-30 | g |
| Soy-based phospholipid | 2-7 | g |
| Propylene glycol | 10-30 | g |
| Fructose (500 mg/ml) | 50 | g |
| Sucralose (5 mg/ml) | 0.5 | g |
| Orange oil (2 mg/ml) | 0.2 | g |
| Clove Oil (1 mg/ml) | 0.1 | g |
| Sodium benzoate (1 mg/ml) | 0.1 | g |
| Water to 100 ml | | |

ethanol and soy lipid are mixed together by slowly adding with stirring and warming;
sodium benzoate is added to this warm mixture;
PG is added to the mixture with stirring;
fructose and sucralose are slowly added with constant stirring;
orange oil and clove oil are added and the mixture is stirred for a further 30 minutes;
the mixture is cooled to 20-25° C.;
nimorazole is added and the suspension is stirred for a further 60 minutes or until homogenous.
pH is adjusted to about 3.5-4.0 with citric acid and the volume is made up to 100 ml with stirring.

### Example 6

### Non-Aqueous Based Suspension with Taste Masking

A sixth oral suspension of a hypoxic radiation sensitiser of the invention is prepared as follows:

| | |
|---|---|
| Nimorazole (250 mg/ml) | 25 g |
| 2,6 diisopropylphenol (10 mg/ml) | 1 g |
| Glycerol 22 mg/ml) | 22 g |
| Egg phospholipid (12 mg/ml) | 12 g |
| Fructose (500 mg/ml) | 50 g |
| Sucralose (5 mg/ml) | 0.5 g |
| Orange oil (2 mg/ml) | 0.2 g |
| Clove Oil (1 mg/ml) | 0.1 g |
| Sodium benzoate (1 mg/ml) | 0.1 g |
| Soya bean oil to 100 ml | |

Glycerol is dissolved in 30 ml soya bean oil by slowly adding with stirring;
egg phospholipid is added to the mixture with stirring;
sodium benzoate is dissolved in warm diisopropylphenol (60-80° C.);
this mixture is added to the glycerol mixture with stirring;
fructose and sucralose are slowly added with constant stirring;
orange oil and clove oil are added and the mixture is stirred for a further 30 minutes;
the mixture is cooled to 20-25° C.;
nimorazole is added and the suspension is stirred for a further 60 minutes or until homogenous;
pH is adjusted to about 3.5-4.0 with citric acid and the volume is made up to 100 ml with stirring.

### B. Examples of Solutions of the Invention

The examples make a solution of the invention as a syrup in a concentration range from 1 mg/ml to 5000 mg/ml. The following are formulations per 100 ml. Colour can be added as required, for example Cochineal Red A as 3 mg per 100 ml.

### Example 7

| | | |
|---|---|---|
| Nimorazole (5 mg/ml) | 0.5 | g |
| Methyl paraben | 100 | mg |
| Propyl paraben | 50 | mg |
| Peppermint oil | 0.5 | ml |
| Sorbitol powder (70% final) | 70 | g |
| Water | to 100 | ml |

Method:
Nimorazole is dissolved in 80 ml of water with heat up to 60° C. and stirring. Parabens' are added and dissolved with the mild heat if necessary;
the composition is allowed to cool to room temperature;
sorbitol powder is added with stirring (and heat if necessary);
the composition is allowed to cool and peppermint oil added;
The volume is made up to 100 ml.

The pH can be adjusted to pH 4-5 which is just below that of the pKa of Nimorazole (pH 5.2) and below that of the bucal cavity of about 6.5 for greater comfort of the patient.

### Example 8

| | | |
|---|---|---|
| Nimorazole (5 mg/ml) | 0.5 | g |
| Methyl paraben | 100 | mg |
| Propyl paraben | 50 | mg |
| Glycerin USP | 10 | ml |
| Polysorbate 80 | 1 | ml |
| Citric acid USP | 0.2 | g |
| Saccharin sodium USP monohydrate | 0.15 | g |
| Raspberry flavour | 3.0 | ml |
| Water | to 100 | ml |

Method:
Parabens' are dissolved in 80 ml of water with heat up to 60° C. and stirring. Citric acid and polysorbate 80 are added and dissolved;
Saccharin is added and stirred to dissolution;
nimorazole is added and dissolved with the mild heat;
the composition is allowed to cool to room temperature;
glycerin is added and then add raspberry flavour;
the volume is made up to 100 ml with stirring;
note that the pH may be adjusted to pH 4-5 which is just below that of the pKa of Nimorazole (pH 5.2) and below that of the bucal cavity of about 6.5.

Saccharin may be replaced by sucralose at 0.5 g/100 ml.

The parabens' may be replaced by sodium benzoate 0.2 g/100 ml. The pH must be adjusted to below pH 4.5 when the sodium benzoate is used, preferably pH 3-4).

### Example 9

### Higher Concentration Syrups

The solubility of nimorazole is significantly increased by complexing with substituted cyclodextrins. A large number of these exist but in this formulation Hydroxypropyl beta Cyclodextrin (HPbCD) is used. HPbCD also assists masking the taste of the drug.

A solubility increase of above 10 fold is achieved and the room temperature solubility of nimorazole may be increased to the more dose convenient level of 100 mg/ml.

HPbCD complexes with nimorazole over a wide molar ratio, in this example the lower ratio of 1:0.1 is used, therefore 226 g of nimorazole is used with 1400 g of HPbCD.

Thus in a syrup of 100 mg/ml nimorazole, the complexing amount of HPbCD would be 579 mg, or in a 100 ml solution, 10 g nimorazole requires 57.9 g of HPbCD.

The formulation below can be scaled as appropriate with the Nimorazole/HPbCD mixture added to the ingredients to 5000 mg/ml. The scale-up only applies to the increase in the 1:1 molar ratio of the powder preparation. The other ingredients do not change. At the 1000 mg/ml level this will involve 100 g of nimorazole per 100 ml, and 579 g of HPbCD

| HPb Cyclodextrin | 57.9 | g |
|---|---|---|
| Nimorazole | 10 g Powder Complex A | |
| Methyl paraben | 100 | mg |
| Propyl paraben | 50 | mg |
| Peppermint oil | 0.5 | ml |
| Sorbitol 70% solution | 90 | ml |
| Water | to 100 | ml |

### Preparation of a Powder Complex A

Preferred method: Kneading preparation

This is the preferred method due to improved dissolution of the drug in humans on ingestion of the final product syrup.
The nimorazole is taken up in a small amount of ethanol and water (1:1), approximately 5-10 ml;
pH can be adjusted to below the Nimorazole pKa of 5.2 with dilute Citric acid—pH 4-5);
the mixture is blended into a paste with the HPbCD for 1-2 hours in a suitable paste blender, kneader or high sheer granulator/blender or a mortar and pestle;
once mixing is complete, the paste is dried at 45° C. and ground to a powder suitable for mixing with Solution B.

The resultant powder above or in any of the methods shown below should have a water content of between 10-12%. Prior to use with Solution B, the water content is determined and the addition of the complex Powder A is adjusted for the water content to obtain a final mixture solution of 100 mg/ml Alternatives to this method involve different ways to bind of complex the Nimorazole with the HPbCD. These may include:
(i) Co-evaporation of the Nimorazole with the HPbCD by taking up the Nimorazole in 10 ml of either pure ethanol or 50% ethanol and water (pH adjusted as above) and adding this to an aqueous solution of the HPbCD (approximately 10 ml). The mixture is stirred for between 2-6 hours, then the solvents evaporated and the powder dried and ground for addition to Solution B.
(ii) Rotary Evaporation of the Nimorazole with the HPbCD by adding the 10 g of Nimorazole and the 57.9 g of HPbCD to 100 ml of water (pH adjusted as above) and stirring at 60° C. for 2 hours.
The mixing occurs in a rotary evaporator and is dried under vacuum overnight at 45° C.
(iii) Ball Milling by adding an amount equivalent of about 300 g (i.e. 40 g Nimorazole and 231.6 g of HPbCD) in two amounts of Nimorazole and HPbCD to a ball mill and blending for 1 hour (500 g capacity ball mill with 13 mm balls at 27 rpm).
(iv) Alternative Kneading Method—one part of water is gradually added for each three parts by weight of HPbCD (57.9 g) while mixing. (The water is pH adjusted as above). The mixing is continued until a homogeneous viscous paste is obtained. If the mixer is not powerful enough more water may need to be added to get a consistent viscous paste. Nimorazole (10 g) is gradually added and mixed at 20° C. or room temperature for 10-60 minutes, once all the drug has been added. An automatic apparatus is used for 10 minutes, and 60 for manual mixing. The resultant blend can be dried in the mixer if it has a heater or in a drying oven from 20-50° C. for 2-10 hours, (preferably 40° C.) followed by grinding and sieving of the powder if necessary. The product should be stored in a dessicator unless it is to be used within 1 hour.

### Preparation of a Solution B

Parabens' are dissolved in 90 ml of the sorbitol solution with mild heat if necessary, and allowed to cool. (The parabens' may be replaced by 10 mg of benzalkonium chloride or by 0.1 g of benzyl alcohol)

### Mixing of Powder Complex A and Solution B

Powder complex A is mixed into 90 ml of Solution B for 30 minutes. Peppermint oil is added and the solution is thoroughly stirred and made up to 100 ml.

pH may be adjusted to pH 4-5 which is just below that of the pKa of Nimorazole (pH 5.2) and below that of the bucal cavity of about 6.5.

### Example 10

This example uses Powder Complex A as described above, ensuring that the moisture level is determined and the addition adjusted for moisture to create a final drug concentration of 100 mg/ml.

| HPb Cyclodextrin | 57.9 | g |
|---|---|---|
| Nimorazole | 10 g Powder Complex A | |
| Methyl paraben | 100 | mg |
| Propyl paraben | 50 | mg |
| Sucralose | 0.5 | g |
| Peppermint oil | 0.5 | ml |
| Water | to 100 | ml |

### Solution B

Parabens' are dissolved into 90 ml of, water with the mild heat if necessary. Acesulfame and sucralose are added and the mixture is allowed to cool. Powder Complex A is stirred into 90 ml of Solution B for 30 minutes. Peppermint oil is added and the mixture is stirred thoroughly and made up to 100 ml.

Note that the pH may be adjusted to pH 4-5 which is just below that of the pKa of Nimorazole (pH 5.2) and below that of the bucal cavity of about 6.5.

### Example 11

This example uses Powder Complex A as described above, ensuring that the moisture level is determined and the addition adjusted for moisture to create a final drug concentration of 100 mg/ml.

| | | |
|---|---|---|
| Nimorazole (100 mg/ml) | 10 | g |
| Fructose (500 mg/ml) | 50 | g |
| Sucralose (5 mg/ml) | 0.5 | g |
| Orange oil (2 mg/ml) | 0.2 | g |
| Clove Oil (1 mg/ml) | 0.1 | g |
| Methyl paraben | 100 | mg |
| Propyl paraben | 50 | mg |
| Water | to 100 | ml |

Method:
Parabens' are dissolved in 80 ml of warm (60° C.) purified water for 30 minutes with stirring;
fructose and sucralose are added slowly with constant stirring until dissolved (about 60 minutes);
nimorazole (Powder Complex A) is added and the solution is stirred for a further 60 minutes or until the drug complex is dissolved. Added heat up to 60° C. may be required;
orange oil and clove oil are added and the mixture is stirred for a further 30 minutes;
pH is adjusted to about 4-5 with citric acid and the volume is made up to 100 ml with stirring.

### Example 12

### Propylene Glycol (PG) Based Solution with Taste Masking

| | | |
|---|---|---|
| Nimorazole (100 mg/ml) | 10 | g |
| Ethanol | 2-30 | g |
| Soy-based phospholipid | 2-7 | g |
| Propylene glycol | 10-30 | g |
| Fructose (500 mg/ml) | 50 | g |
| Sucralose (5 mg/ml) | 0.5 | g |
| Orange oil (2 mg/ml) | 0.2 | g |
| Clove Oil (1 mg/ml) | 0.1 | g |
| Methyl paraben | 100 | mg |
| Propyl paraben | 50 | mg |
| Water | to 100 | ml |

Method:
ethanol and soy lipid are mixed together by slowly adding with stirring and warming;
the parabens' are added to this warm mixture and stirred to dissolution;
PG is added to the mixture with stirring;
fructose and sucralose are slowly added with constant stirring;
orange oil and clove oil are added and the mixture is stirred for a further 30 minutes and the mixture is cooled to 20-25° C.;
nimorazole is added and the solution is stirred for a further 60 minutes or until homogenous. Mild heat to 60° C. may be required;
pH is adjusted to about 4-5 with or citric acid and the volume is made up to 100 ml with stirring.

### Example 13

### Non-Aqueous Based Solution with Taste Masking

| | | |
|---|---|---|
| Nimorazole (100 mg/ml) | 10 | g |
| 2,6 diisopropylphenol (10 mg/ml) | 1 | g |
| Glycerol 22 mg/ml) | 22 | g |
| Egg phospholipid (12 mg/ml) | 12 | g |
| Fructose (500 mg/ml) | 50 | g |
| Sucralose (5 mg/ml) | 0.5 | g |
| Orange oil (2 mg/ml) | 0.2 | g |
| Clove Oil (1 mg/ml) | 0.1 | g |
| Methyl paraben | 100 | mg |
| Propyl paraben | 50 | mg |
| Soya bean oil | to 100 | ml |

Method:
Glycerol is dissolved in 30 ml soya bean oil by slowly adding with stirring;
egg phospholipid is added to the mixture with stirring;
the parabens are dissolved in warm diisopropylphenol (60-80° C.) with stirring;
this mixture is added to the glycerol mixture with stirring;
fructose and sucralose are slowly added with constant stirring;
orange oil and clove oil are added and the mixture is stirred for a further 30 minutes;
nimorazole is added and the solution is stirred for a further 60 minutes or until homogenous, Mild heat to 60 degrees may be required;
pH is adjusted to about 4-5 with citric acid and the mixture is cooled to 20-25° C.;
the volume is made up to 100 ml with stirring.

### Example 14

### Aqueous Based Salt Solution Without Taste Masking

This example is of a non taste masked solution of the invention as a syrup in a concentration range from 1 mg/ml to 5000 mg/ml. The following is a formulation per 100 ml. Colour can be added as required, for example Cochineal Red A as 3 mg per 100 ml.

| | | |
|---|---|---|
| Nimorazole Hydrochloride (100 mg/ml) | 10.0 | g |
| Methyl paraben | 100 | mg |
| Propyl paraben | 50 | mg |
| Peppermint oil | 0.5 | ml |
| Sorbitol powder (70% final) | 70 | g |
| Water | to 100 | ml |

Method:
Nimorazole Hydrochloride is dissolved in 80 ml of water with stirring;
Parabens' are added and dissolved with the mild heat if necessary;
the composition is stirred at room temperature for a period of 20 minutes;
sorbitol powder is added with stirring (and heat if necessary);
the composition is allowed to cool and peppermint oil added;
The volume is made up to 100 ml.

The pH can be adjusted to pH 3-4, below that of the bucal cavity.

### Example 15

### Aqueous Based Salt Solution with Taste Masking

This example is of a taste masked solution of the invention as a syrup in a concentration range from 1 mg/ml to 5000 mg/ml. The following is a formulation per 100 ml. Colour can be added as required, for example Cochineal Red A as 3 mg per 100 ml.

| | | |
|---|---|---|
| Nimorazole Hydrochloride (100 mg/ml) | 10.0 | g |
| Methyl paraben | 100 | mg |
| Propyl paraben | 50 | mg |
| Sucralose | 500 | mg |
| Polysorbate 80NF | 1.0 | ml |
| Glycerin USP | 10 | ml |
| Citric Acid | 200 | mg |
| Raspberry flavour | 3 | ml |
| Water | to 100 | ml |

Method:
Nimorazole Hydrochloride is dissolved in 80 ml of water with stirring;
Parabens' are added to this solution with mild heat and stirring to dissolve them and then cooled;
citric acid is added and the solution is stirred;
glycerin is added with stirring;
polysorbate 80 is added and the solution is stirred;
sucralose is added and the solution is stirred;
raspberry flavour is added and the solution is stirred for a period of 20 minutes;
The volume is made up to 100 ml.
   The pH can be adjusted to pH 3-4, below that of the bucal cavity.

It is clear from the foregoing that oral liquid compositions of the invention provide an improved composition and method of administration of radiation sensitisers to patients undergoing radiotherapy. The oral liquid compositions of the invention overcome the problem of administration of many large doses of the radiation sensitiser as a tablet or capsule. The oral liquid compositions of the invention also increase patient comfort and compliance.

Throughout the specification the aim has been to describe the preferred embodiments of the invention without limiting the invention to any one embodiment or specific collection of features.

Throughout this specification, unless the context requires otherwise, the word “comprises”, and variations such as “comprise” or “comprising”, will be understood to imply the inclusion of a stated integer or group of integers or steps but not to the exclusion of any other integer or group of integers.

1. An oral liquid composition of a hypoxic radiation sensitiser comprising the radiation sensitiser in a concentration of greater than 5 mg/ml.

2. The oral liquid composition of claim 1 wherein the hypoxic radiation sensitiser is a nitroimidazole or salt thereof.

3. The oral liquid composition of claim 1 wherein the radiation sensitiser is nimorazole or a salt thereof, or functional derivative of nimorazole.

4. The oral liquid composition of claim 1 wherein the radiation sensitiser is selected from the group consisting of nimorazole hydrochloride, nimorazole maleate, nimorazole tosylate, and nimorazole fumarate.

5. The oral liquid composition of claim 1 wherein the radiation sensitiser is selected from the group consisting of nimorazole succinate, nimorazole sulphate, and nimorazole mesylate.

6. The oral liquid composition of claim 1 wherein the radiation sensitiser is selected from the group consisting of nimorazole benzoate, nimorazole adipate, nimorazole citrate, nimorazole gentisate, nimorazole hippurate, nimorazole lactate, nimorazole phosphate, and nimorazole saccharinate.

7. The oral liquid composition of claim 1 wherein the radiation sensitiser is selected from the group consisting of nimorazole ascorbate, nimorazole glutamate and nimorazole naphthalate.

8. The oral liquid composition of claim 1 wherein the oral liquid composition is an aqueous oral liquid composition.

9. The oral liquid composition of claim 1 wherein the oral liquid composition is a non-aqueous oral liquid composition.

10. The oral liquid composition of claim 1 wherein the oral liquid composition includes one or more additional pharmaceutically active agents.

11. The oral liquid composition of claim 1 wherein the oral liquid composition includes taste-masking, solubilising and/or preservative agents.

12. The oral liquid composition of claim 1 the foregoing claims further comprising at least one paraben.

13. A method of treating a patient suffering from a hypoxic cancer comprising oral administration of a liquid composition of a hypoxic radiation sensitiser and radiotherapy to the patient, wherein the liquid composition comprises the radiation sensitiser in a concentration of greater than 5 mg/ml.

14. Use of an oral liquid composition of a hypoxic radiation sensitiser of concentration greater than 5 mg/ml in conjunction with radiotherapy to treat hypoxic cancer.

15. The method of claim 14 wherein the hypoxic radiation sensitiser is a nitroimidazole or salt thereof, or functional derivative of nimorazole.

16. The method of claim 15 wherein the radiation sensitiser is selected from the group consisting of nimorazole hydrochloride, nimorazole maleate, nimorazole tosylate, nimorazole fumarate, nimorazole succinate, nimorazole sulphate, and nimorazole mesylate, nimorazole benzoate, nimorazole adipate, nimorazole citrate, nimorazole gentisate, nimorazole hippurate, nimorazole lactate, nimorazole phosphate, nimorazole saccharinate, nimorazole ascorbate, nimorazole glutamate and nimorazole naphthalate.

17. The method of claim 14 wherein the oral liquid composition is an aqueous oral liquid composition.

18. The method of claim 14 wherein the oral liquid composition is a non-aqueous oral liquid composition.

19. The method of claim 14 wherein the oral liquid composition includes one or more additional pharmaceutically active agents.

20. The method of claim 14 wherein the oral liquid composition further comprises at least one paraben.
